(19) United States
(12) Patent Application Publication
(10) Pub. No.: US 2003/0170299 A1
(43) Pub. Date: Sep. 11, 2003

(54) Therapeutic methods for acute myeloid leukemia

(76) Inventors: Robert J. Lee, Columbus, OH (US); Manohar Ratnam, Toledo, OH (US)

Correspondence Address: CALFEE HALTER & GRISWOLD, LLP, 800 SUPERIOR AVENUE, SUITE 1400, CLEVELAND, OH, 44114, US

(21) Appl. No.: 10/375,888

(22) Filed: Feb. 27, 2003

Related U.S. Application Data
(60) Provisional application No. 60/360,408, filed on Feb. 27, 2002.

(51) Int. Cl. A61K 31/525; A61K 9/127; A61K 31/203

(52) U.S. Cl. 424/450; 514/251; 514/559

(57) ABSTRACT

The invention provides a method for treating leukemia in a patient. The method comprises administering to the patient a substance that increases expression of folate receptor β on leukemia cells in the patient, called a FR-β inducer, and administering a folate-conjugated therapeutic that targets the leukemia cells in the patient. The invention also comprises pharmaceutical compositions containing one or both of a FR-β inducer and a folate-conjugated therapeutic. The invention also provides a kit for use in treating leukemia in a patient, the kit comprising an FR-β inducer and a folate-conjugated therapeutic

This application claims priority from U.S. Provisional Patent Application Serial No. 60/360,408, filed on Feb. 27, 2002, which is incorporated herein by reference. This invention was made, at least in part, with government support under National Institutes of Health R01 Grants CA80183 and CA70873. The U.S. government has certain rights in the invention.

### FIELD OF THE INVENTION

This invention relates to methods for treating leukemia. More specifically, this invention relates to methods for treating patients with myeloid leukemia, preferably acute myelogenous leukemia (AML), by administering agents that increase levels of folate receptor β (FR-β), and then treating the patient with folate-conjugated anticancer therapeutic agents.

### BACKGROUND

Leukemias are neoplastic disorders involving cells of the blood-forming organs. Leukemias are commonly classified as either myeloid or lymphoid. Myeloid leukemias involve the myeloid elements of the bone marrow—white cells, red cells and megakaryocytes. Myeloid leukemia, which accounts for half of all leukemia cases, is classified as acute myelogenous leukemia (AML) or chronic myelogenous leukemia (CML). AML is the most common form of leukemia in adults and is classified according to the French-American-British (FAB) criteria into classes M0-M7 based on the degree of differentiation and the extent of cell maturation. Standard AML chemotherapy, which often includes an anthracycline, results in a 70% complete remission (CR) rate in AML patients. Anthracycline therapy, however, is associated with severe side effects, including myelosuppression and dose-limiting cardiotoxicity, as well as a significant incidence of relapse. Less than 20% of CR patients survive in the long term.

Relapsed AML disease exhibits multiple drug resistance (MDR), making the relapsed disease frequently refractory to further treatment with a variety of chemotherapeutic agents, including drugs. In leukemia, MDR is most frequently associated with a 170-kDa transmembrane glycoprotein encoded by the MDR-1 gene, known as the permeability glycoprotein (P-gp). P-gp occurs in 30-50% of AML patients, with a higher percentage in those patients with relapsed or chemo-refractory disease. Combining anti-leukemic drugs with a P-gp modulator has had limited success in overcoming this problem, due to both the dose-limiting toxicity of the modulator and the toxicity resulting from reduced drug excretion. Therefore, new treatments should not only be selective for AML, but also bypass MDR.

For a subset of AMLs, other treatments have been found. For example, acute promyelocytic leukemia (PML), which accounts for 10% of AMLs, is associated cytogenetically with a t(15;17) chromosomal translocation, which results in production of a PML-retinoic acid receptor alpha (RARα) gene fusion product. Complete remission can be achieved in 72-95% of PML patients using all-trans retinoic acid (ATRA or tretinoin), which causes differentiation of the PML cells. However, this therapy is also followed by a significant incidence of relapse. Patients with relapsing PML disease are often refractory to further ATRA therapy due to mutations that block retinoid-induced differentiation. The 90% of AMLs that are not PML do not respond to ATRA therapy at all.

In an attempt to address these problems, there has been significant work to develop targeted therapeutic agents for myeloid leukemias. Such targeted therapeutics most often consist of a ligand with affinity for a molecule on the surface of the tumor cell of interest, coupled to a therapeutic drug, most often a cytotoxic agent. Binding of the ligand to the surface molecule results in high concentrations of the cytotoxic agent being brought to the tumor cell. For such therapeutics to be effective, there is specificity of the agent for tumor cells relative to other, non-tumor cells.

Examples of targeted therapeutics include Gemtuzumab ozogamicin (CMA-676), which consists of a cytotoxic drug, calichaemicin, linked to a human monoclonal antibody (huMAb) specific for the myelocyte marker CD33, has recently been approved by the FDA for clinical use. Gleevec™, a bcr-abl tyosine kinase inhibitor, is 90% effective against CMLs and causes minimal side effects. Targeted immunotoxins and cytokine fusion toxins for treatment of AMLs have also been developed. These include anti-CD64-Ricin A Chain, anti-CD7 and anti-CD38-Saporin, anti-Tac(Fv)-Pseudomonas exotoxin (PE38) (LMB-2), anti-CD33 gelonin, and granulocyte-macrophage colony-stimulating factor (GM-CSF) fusion proteins with Pseudomonas exotoxin (PE38) and diphtheria toxin. All of these therapies have their particular strengths and weaknesses.

One cell surface molecule of interest for which targeted therapeutics have been developed is the folate receptor (FR). Three human isoforms of FR exist: FR-α, FR-β and FR-γ. FR-α and FR-β are attached to the cell surface through a glycosyl-phosphatidylinositol (GPI) membrane anchor. FR-γ, is secreted and, therefore, is not targetable by such therapeutics.

Expression of FR-α in normal tissues is restricted to the luminal surface of certain epithelial cells, where it is inaccessible via the circulation. FR-α is overexpressed in certain carcinomas, particularly in major gynecological tumors such as ovarian cancer, where it is accessible for tumor-selective targeting through the bloodstream.

FR-β is normally found in placental tissues and in hematopoietic cells where it is expressed in the myelomonocytic lineage and is particularly elevated during neutrophil maturation or during monocyte or macrophage activation. However, the FR-β expressed on normal hematopoietic cells, unlike that on activated macrophages for example, is nonfunctional in that it cannot bind and internalize folate. FR-β is expressed on malignant cells from patients with CML, and on malignant cells from approximately 70% of patients with AML.

There continues to be a need for new targeted therapeutics for myeloid leukemias, and for methods of treating individuals with myeloid leukemias, specifically AML, using effective therapies that are specifically targeted to mycloid leukemia cells, have low toxicity and can be used for treatment of patients that have developed resistance to other methods and therapeutic agents.

### SUMMARY OF THE INVENTION

The present invention provides methods for treating patients having myeloid leukemias, preferably AML. The method comprises administering to a patient, an amount of an FR-β inducer sufficient to increase the level of FR-β on the plasma membrane of myeloid leukemia cells, including blast cells, progenitor cells, and stem cells, and administering a biologically effective amount of a folate-conjugated therapeutic, comprising a cytotoxic drug, to the patient.

We have found that expression of FR-β is increased in malignant cells from myeloid leukemia patients by FR-β inducers. In one embodiment, the FR-β inducer is an agonist of one or more of the retinoic acid receptors (RAR) alpha, beta, or gamma. One particularly useful agonist is all-trans retinoic acid (ATRA). In another embodiment, a retinoic acid receptor agonist and a histone deactylase inhibitor are administered to the patient. One particularly suitable histone deactylase inhibitor is Trichostatin A (TSA).

We have also found that the FR-β expressed in myeloid leukemia cells, preferably AML cells, is functional in that it binds and internalizes folate, unlike the FR-β expressed in the majority of normal hematopoietic cells which is nonfunctional. Such functional FR-β is a target for folate-conjugated therapeutics of the present invention. In one embodiment, the folate-conjugated therapeutic is a liposome with folate attached to or conjugated to the liposome, the liposome containing or associated with a therapeutic drug, preferably a drug cytotoxic for tumor cells. Preferably, folate is covalently attached to a molecule that is a part of the lipid bilayer of the liposome. Preferably, folate is attached to a lipid that is part of the liposome through a flexible hydrophilic polymer, such as polyethylene glycol (PEG).

### BRIEF DESCRIPTION OF THE DRAWINGS

FIG. 1. Expression and properties of FR-β in peripheral blood granulocytes and in recombinant CHO-FR-β cells. A. FR-β expression detected in cell lysates of neutrophils, and control Chinese hamster ovary cells expressing a transfected FR-β gene, by Western blot using anti-FR-β antiserum. The band intensities were estimated by the NIH Image software. B. Differential sensitivity of FR-β GPI anchors to PI-PLC and nitrous acid; whole cells were treated with PI-PLC and the cell lysates analyzed by Western blot using anti-FR-β antibody; alternately, cell membranes were treated with nitrous acid and similarly analyzed. C. Cellular binding of FITC-folate determined by flow cytometry. Ten nM FITC-folate was used with or without pre-incubation with 1 μM unlabeled folic acid.

FIG. 2. Structure of Folate-PEG-DSPE.

FIG. 3. Fluorescence micrographs of KG-1 cells treated with calcein-containing liposomes. KG-1 cells were incubated with f-L-calcein (left) or L-calcein (right) for 1 hour at 37° C. and washed 3 times with cold PBS. The images of the live cells were collected with a digital imaging system.

FIG. 4. Dose response and time course of FR-β induction by ATRA in KG-1 cells. A. Dose response by Western blot with an anti-FR-β antibody and AP-conjugated secondary Ab. B. Dose response by flow cytometry using anti-FR-β antibody and FITC-conjugated secondary Ab. C. Time dependence by flow cytometry. D. Response to ATRA removal by Western blot: lane 1, without ATRA; lane 2, with 1 μM ATRA for 5 days; lane 3, 5 days with 1 μM ATRA followed by 7 days without ATRA.

FIG. 5. ATRA induction of FR-β in AML blasts from patient marrow. Cells (1×10⁶) were cultured and treated with or without ATRA (1 μmol/L) for 6 hours (A and B), 24 hours (C), or 5 days (D). The samples were obtained from separate patients with FAB-M2 (A and C) or FAB-M4 (B and D) type AML. Cell lysates were then examined by Western blot and the use of rabbit anti-FR-β- and AP conjugated antibodies.

FIG. 6. The effect of ATRA on FR-β expression and f-L-calcein uptake in KG-1 cells. Cellular FR-β expression was determined by flow cytometry using rabbit anti-FR-β (with normal rabbit IgG as an isotype control) as the primary antibody and FITC-goat-anti-rabbit IgG as the secondary antibody. Liposomal uptake was determined by fluorescence of the encapsulated calcein. A. FR-β expression in untreated KG-1 cells; B. FR-β expression in KG-1 cells after a 5-day exposure to ATRA (1 μM); C. Uptake of f-L-calcein and L-calcein by KG-1 cells and the effect of ATRA. The cells were treated with L-calcein without ATRA pre-treatment (1); L-calcein with ATRA pre-treatment (2); f-L-calcein without ATRA pre-treatment (3); or f-L-calcein with ATRA pre-treatment (4).

FIG. 7. Tumor cell uptake of [^{99m}Tc]HYNIC-folate. Top: structure of the radioconjugate [^{99m}Tc]HYNIC-folate; Bottom: concentration-dependent cellular binding.

FIG. 8. A γ-camera image of a C57BL/6 mouse carrying syngeneic subcutaneous FR(+) tumors injected intravenously with ^{99m}Tc-HYNIC-folate. The image was obtained at 4 hour post-injection.

FIG. 9. FR-Mediated Uptake of ¹¹¹In-labeled folate-PEG-liposomes by cultured KB cells. A. Structure of folate-PEG-Chol. B. KB cell uptake of I) non-targeted control liposomes; II) targeted liposomes containing 1 mole % folate-PEG-DSPE; III) same as II except in the presence of 1.5 nM free folate; IV) targeted liposomes containing 1 mole % folate-PRG-Chol; and V) same as IV) except in the presence of 1.5 nM free folate. C. Competitive inhibition of FR-binding by free folate.

FIG. 10. Effect of f-L-DOX treatment on the survival of DBA/2 mice carrying L1210JF ascites tumor. DBA/2 mice inoculated intraperitoneally with L1210JF cells that were treated with saline, free DOX, L-DOX or f-L-DOX. Animal survival was recorded starting from the day of tumor cell inoculation.

FIG. 11. Effect of f-L-DOX treatment on the survival of SCID mice carrying KG-1 ascites tumor. SCID mice inoculated intraperitoneally with KG-1 cells that were treated with saline, free DOX, L-DOX or f-L-DOX, with or without co-injection of ATRA. Animal survival was recorded starting from the day of tumor cell inoculation.

FIG. 12. Scheme for synthesis of folate-γ-PEG3,350-DSPE.

FIG. 13. Effect of TSA concentration on reporter gene expression in stably transfected 293 cells treated with both TSA and ATRA. 293 cells that were stably transfected with an FR-β promoter-luciferase reporter construct were treated with 1 μM ATRA and varying concentrations of TSA for 5 days. The cells were then harvested and tested for luciferase expression, which indicates FR-β promoter activity.

FIG. 14. Effect of ATRA concentration on reporter gene expression in stably transfected 293 cells treated with both TSA and ATRA. 293 cells that were stably transfected with an FR-β promoter-luciferase reporter construct were treated with 30 ng/ml TSA 1 and varying concentrations of ATRA for 5 days. The cells were then harvested and tested for luciferase expression, which indicates FR-β promoter activity.

FIG. 15. Effect of TSA concentration on reporter gene expression in KG-1 human AML cells treated with both TSA and ATRA. Human AML KG-1 cells were treated with 1 μM ATRA and varying concentrations of TSA for 5 days. Cells were harvested and total mRNA was purified. The expression of FR-β mRNA was determined by real time RT-PCR.

FIG. 16. Effect of ATRA concentration on reporter gene expression in KG-1 human AML cells treated with both TSA and ATRA. KG-1 cells were treated with 1 μM ATRA and different concentrations of TSA for 5 days. Cells were harvested and membranes were isolated. The expression of FR-β protein was then tested by Western blot. The lanes of the figure are as follows: 1, untreated; 2, 1 μM ATRA; 3, 10 ng/ml TSA; 4, 1 μM ATRA+10 ng/ml TSA; 5, 30 ng/ml TSA; 6, 1 μM ATRA+30 ng/ml TSA; 7, 50 ng/ml TSA; 8, 1 μM ATRA+50 ng/ml TSA.

### DETAILED DESCRIPTION OF THE INVENTION

Definitions

Herein, “leukemia cells,” “AML cells,” and “CML cells” refer to malignant cells from a patient that has the particular disease, leukemia, AML, and CML, respectively.

Herein, “FR-β inducer,” refers to substances that, when administered to a patient with leukemia, causes an increase in the amount of folate receptor-β (FR-β) on the leukemia cells in the patient.

Herein, “folate-conjugated therapeutic” refers to a substance that has at least one folate molecule and at least one associated therapeutic substance, compound, drug or carrier. The therapeutic substance may be a toxin, enzyme, antibody, radiopharmaceutical, or other substance. The therapeutic substance is preferably a cellular cytotoxic agent. The folate-conjugated therapeutic may comprise a molecule in which folate is attached to the therapeutic substance or, alternatively, may comprise a molecule in which the folate is indirectly attached to or associated with the therapeutic substance through a carrier, such as a liposome or other biocompatible particle. For example, the therapeutic substance may be attached to or contained within a particle, polymer, or other biocompatible material. The particle, polymer or biocompatible material is attached to at least one of the folate molecules of the folate-conjugated therapeutic. The therapeutic substance is preferably attached to or contained within a liposome. Attachment of the therapeutic substance, particle, polymer, other biocompatible material or liposome to folate may be direct or may be through another, preferably hydrophilic, substance. In the preferred embodiment, for example, liposomes of the folate-conjugated therapeutic are attached to folate through polyethylene glycol (PEG). Liposomes that are attached to folate, whether the folate is attached directly to the liposomes, or through a molecule such as PEG, are called “folate-coated liposomes.”

The present invention provides for methods of treating a patient that has leukemia, preferably myelogenous leukemia, more preferably acute myelogenous leukemia (AML). The method comprises administering to the patient, a substance that causes increased expression of folate receptor β (FR-β) on malignant cells of the cancer in the patient. Such a substance is called an FR-β inducer.

The method also comprises administering to the patient, a folate-conjugated therapeutic which comprises folate associated with a substance that is cytotoxic to, or at least inhibits growth of, the malignant cells of the leukemia that express FR-β in the patient. The folate part of the folate-conjugated therapeutic binds to the FR-β of the leukemia cells in the patient. This binding by the folate part brings the cytotoxic part of the folate-conjugated therapeutic into close proximity with the leukemia cells of the patient. Preferably, the leukemia cells internalize the cytotoxic part of the therapeutic. The cytotoxic part of the therapeutic agent preferably kills the leukemia cells.

The methods have a number of advantages. One advantage is specificity of the treatment for the leukemia cells of the patient relative to cells that are not leukemia cells. For example, FR-β on normal hematopoietic cells does not bind folate, whereas FR-β on leukemia cells does bind and internalize folate. In addition, the methods of the present invention provide for increasing the levels or amount of FR-β on leukemia cells of the patient using substances called. One preferred class of FR-β inducers includes retinoic acid receptor agonists. One preferred retinoid acid receptor agonist is all-trans retinoic acid (ATRA).

Another advantage of the present methods is that they are effective in certain instances where traditional therapies are not. For example, induction of increased levels of functional FR-β by ATRA occurs even in AML cells that do not differentiate in response to ATRA (i.e., cells that are refractory to ATRA differentiation therapy for AML). ATRA induction of FR-β occurs in all AML subtypes. Another example of the effectiveness of the present invention is in the case where the leukemia cells of the patient have acquired multiple drug resistance (MDR) due to P-gp encoded by the MDR-1 gene. Such leukemia cells are resistant to treatment using free doxorubicin (DOX). Using one embodiment of the present invention, specifically a folate-conjugated therapeutic comprising folate-coated liposomes, the liposomes containing DOX, the MDR phenotype of the leukemia cells is bypassed (i.e., such leukemia cells are not resistant to DOX introduced using folate-coated liposomes). Also, folate-coated liposomes are not immunogenic.

The invention, and the findings on which it is based, are described in more detail below.

Non-Functioning of FR-β in Normal Hematopoietic Cells

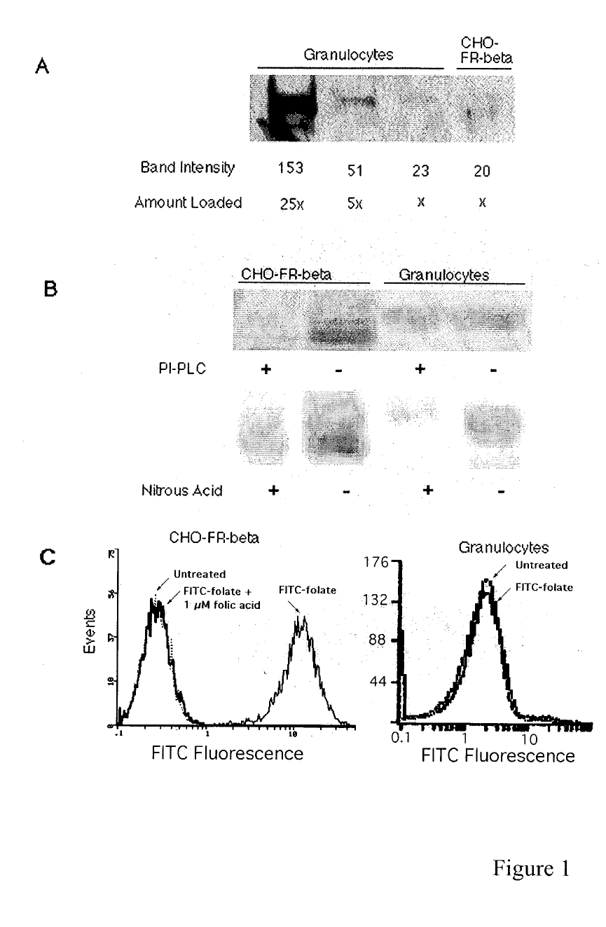

Consistent with other studies, we have shown that FR-β in normal hematopoietic cells does not bind and internalize folate. Since neutrophils express the highest level of FR-β in normal hematopoeisis, the ligand binding property in those cells was examined in one study. Cell lysates were made from normal peripheral blood neutrophils. The cell lysates were subjected to Western or immunoblotting analysis using an antiserum generated against purified FR-β. The results (FIG. 1A) showed a diffuse band with an apparent molecular weight of approximately the same size as FR-β from recombinant CHO-FR-β cells (Chinese hamster ovary cells expressing a transfected FR-β gene). The level of FR-β expressed in neutrophils was comparable to that in the recombinant CHO-FR-β cells.

Functioning of FR-β on the neutrophils was tested with a commonly used diagnostic test for the GPI membrane anchor of FR-β which involved treating cells with phosphatidylinositol-specific phospholipase C (PI-PLC) to release GPI-anchored FR-β from the cells. The results (FIG. 1B) showed that, in contrast to the FR-β in CHO-FR-β cells, the receptor in the neutrophils could not be released from the cell surface with PI-PLC. Several tissues are known to confer modification on the inositol ring of the GPI anchor, causing resistance to PI-PLC. In such instances, the anchor may be cleaved by nitrous acid. Therefore, membrane preparations from the cells were also treated with freshly prepared nitrous acid to cleave the GPI anchor. The results (FIG. 1B) showed that, when membranes prepared from the cells were treated with nitrous acid, FR-β was released, indicating the presence of a modified GPI anchor for FR-β in these cells.

To test whether FR-β on the neutrophils was able to bind folate, the cells were incubated with 10 nM of fluorescein-(FITC) conjugated folate in PBS at 4° C. for 30 minutes in the presence or absence of 1 μM unlabeled folic acid as competitor. The fluorescence of cell surface-bound FITC was measured by flow cytometric analysis. The results (FIG. 1C) showed that FR-β in neutrophils was unable to bind FITC-folate, unlike the FR-β in CHO-FR-β cells.

In an additional study, the nucleotide sequence of cDNA from the neutrophils was determined. No sequence divergence was observed from the known cDNA sequence of FR-β. The data showed, therefore, that FR-β in normal hematopoietic cells, specifically neutrophils, is not functional in binding folate.

Functioning of FR-β in AML Cells

As discussed in the Background section, FR-β is expressed on malignant cells from CML patients and on cells from approximately 70% of AML patients. In one study, we analyzed AML cells from 78 patients for FR-β expression. Table 1 below is a summary of this study, showing FR-β expression in AML cells obtained from patient bone marrow and analyzed by immunofluorescence flow cytometry. CD34 expression on the cells is also noted. The results showed that the AML cells from the majority (68%) of the patients were positive for FR-β. Furthermore, in 72% of the patients who had AML cells that were positive for FR-β (38 out of 53 patients), 100% of the AML cells expressed FR-β. In addition, 66% of the AML cells that were positive for FR-β were also CD34+.

TABLE 1
Expression of FR-β and CD34 on cells from bone marrow aspirates
from patients with AML as determined by flow cytometry

| FAB Class (Total number) | FR-β(+) | FR-β(+)/CD34+ |
|---|---|---|
| M0 (1) | 0 | 0 |
| M1 (9) | 3 | 2 |
| M2 (10) | 4 | 4 |
| M3 (4) | 3 | 0 |
| M4 (13) | 11 | 9 |
| M5 (11) | 11 | 6 |
| M6 (2) | 2 | ND |
| ND (28) | 19 | 14 |

ND = Not Determined
Total No. of AML samples tested: 78
No. of samples in which 100% of the cells were FR-β(+): 38
No. of samples in which 80-90% of the cells were FR-β(+): 4
No. of samples in which 30-60% of the cells were FR-β(+): 8
Total No. of FR-β (+) AML samples: 53; % of FR-β (+) samples: 68

Additionally, we have found that FR-β (+) AML cells bound [³H] folic acid to the extent predicted by the relative level of FR-β expression from flow cytometry using anti-FR-β antibody (0.72±0.13 pmoles per 10⁶ cells). Furthermore, the binding of [³H] folic acid by the FR-β (+) AML cells could be blocked by pre-incubation with 10 nM folic acid, indicating that the binding was FR specific

FR-β Inducers

We have also found that FR-β inducers increase the level of FR-β on the plasma membrane of leukemia cells, including blast cells, progenitor cells, and stem cells. One such group of FR-β inducers is retinoic acid receptor agonists. Such retinoic acid receptor agonists include substances that are agonists of one or more of retinoic acids receptors α, β, and γ. A particularly useful retinoic acid receptor agonist is all-trans retinoic acid (ATRA). Other useful retinoic acid receptor agonists are known in the art as tetramethyl napthalenyl propenyl benzoic acid (TTNPB), 9-cis retinoic acid (9-cis RA), CD336, LG101093 and CD2781, and others. Additional agonists are described in Wang, Zheng, Behm and Ratnam, 2000, Blood 96:3529-3536.

In another embodiment, a retinoic acid receptor agonist and a histone deacetylase inhibitor are administered to the patient. One particularly useful histone deacetylase inhibitor is Trichosatatin A (TSA). Other useful histone deacetylase inhibitors are described in Marks, Richon, Breslow and Rifkind, 2001, Curr. Opin. Oncol. 13:477-83; Yoshida, Furumai, Nishiyama, Komatsu, Nishino and Horinouchi, 2001, Cancer Chemother. Pharmacol. 48 Suppl 1:S20-6; and Jung, 2001, Curr. Med. Chem. 8:1505-11. The FR-β inducers may comprise RAR agonists, alone or in combination with histone deacetylase inhibitors.

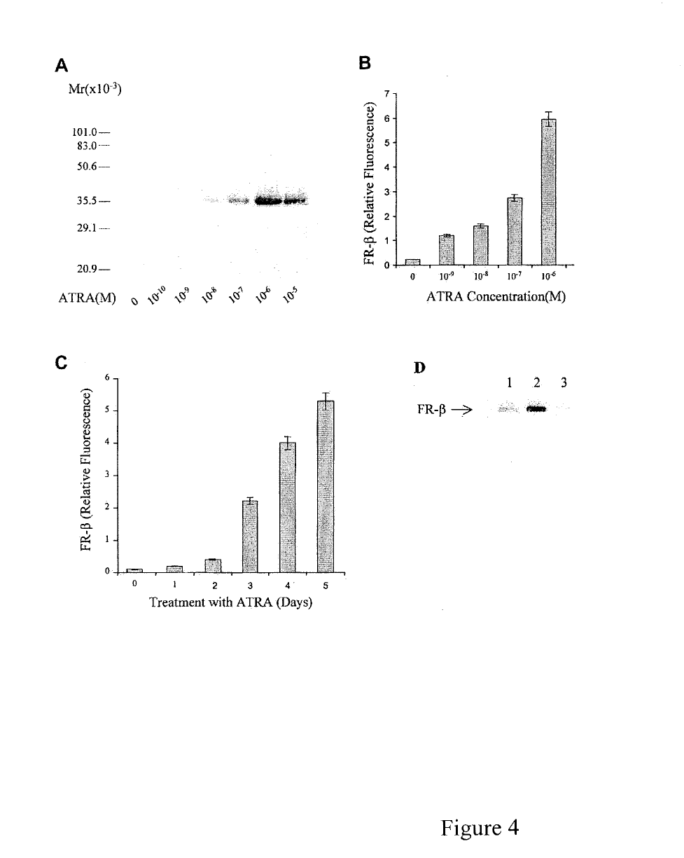

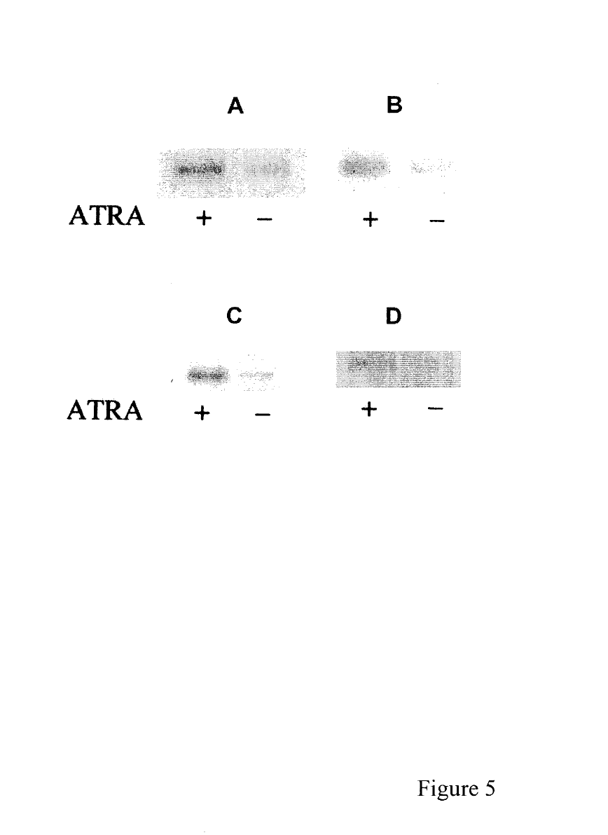

In one study, FR-β expression was elevated up to 20-fold by ATRA in KG-1 myeloid leukemia cells in a dose-dependent and reversible manner in the absence of terminal differentiation or cell growth inhibition (FIG. 4). The retinoid-induced increase in FR-β expression occurred in these cells without causing terminal differentiation or growth inhibition of the cells. In addition, ATRA increased FR-β expression in vitro in myeloid leukemia cells from patient bone marrow (FIG. 5). ATRA-induction of FR-β occurred even though primary AML blast cells (M2 and M4 type AMLs) are known to be refractory to ATRA differentiation therapy. These results indicated that, in order to facilitate FR-targeted therapies, FR-β inducers can be used to increase the levels of FR-β. In addition, FR-β inducers can modulate FR-β expression in AML cells refractory to retinoid differentiation therapy.

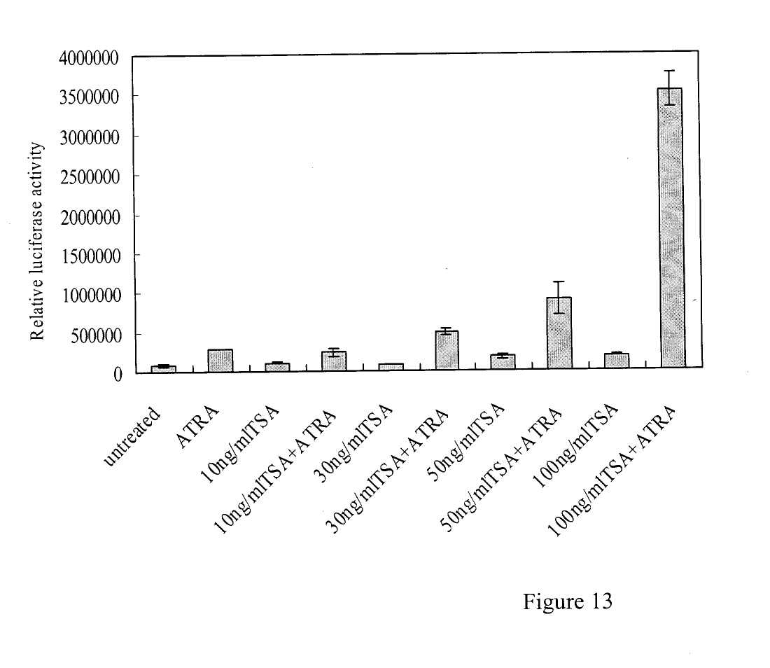

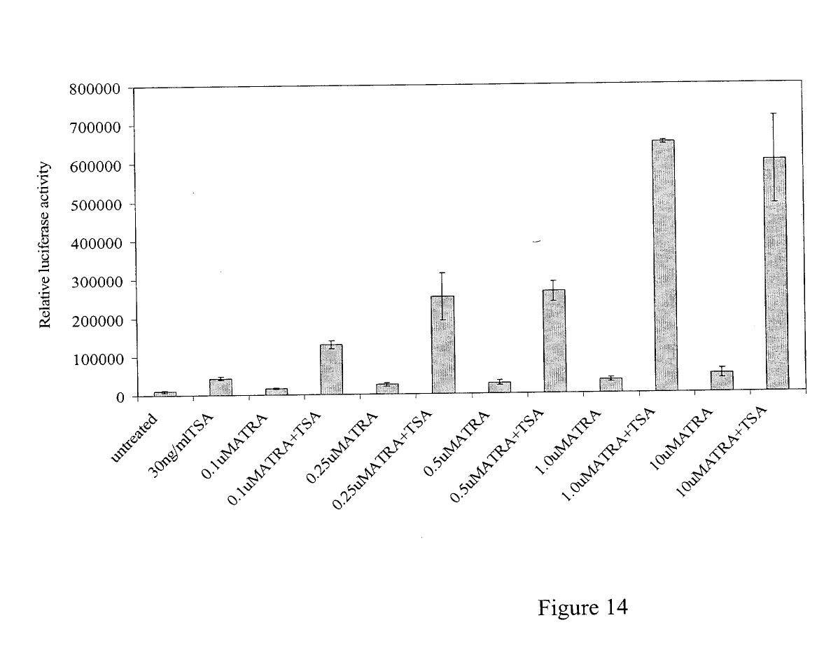

In other studies, the effect of ATRA and the histone deacetylase inhibitor, Trichosatatin A (TSA), on upregulation of FR-β in cultured cells was tested. In one set of studies, 293 cells that were stably transfected with a DNA construct consisting of a luciferase gene whose transcription was regulated by a FR-β promoter. Analysis of luciferase levels in these cells is indicative of the transcriptional activity of the FR-β promoter. Activity of the FR-β promoter is indicative of levels of FR-β in a cell. The data from these studies (shown in FIGS. 13 and 14) showed that there was a synergistic effect between ATRA and TSA such that activity of the FR-β promoter in the presence of ATRA and TSA was greater than activity of the FR-β promoter in the presence of either ATRA or TSA alone.

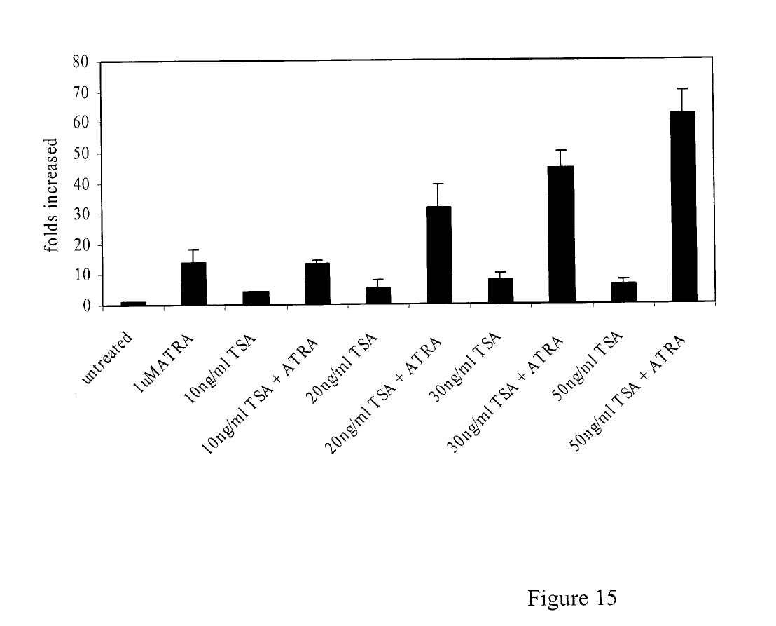

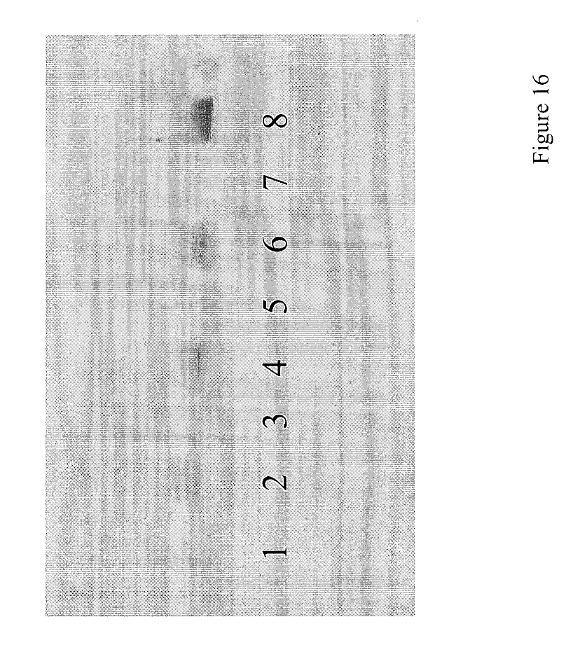

In a second set of studies, human AML KG-1 cells, which are positive for FR-β, were treated with ATRA, TSA or ATRA plus TSA, and then tested for levels of FR-β mRNA using real-time PCR (FIG. 15) and FR-β protein using Western blotting (FIG. 16). The data again showed a synergistic effect between ATRA and TSA such that levels of FR-β in the presence of ATRA and TSA was generally greater than levels of FR-β in the presence of either ATRA or TSA alone. These data indicate that the combination of a retinoic acid receptor agonist and a histone deacetylase inhibitor are advantageous to increase the levels of FR-β on leukemia cells.

Folate-Conjugated Therapeutics for Targeting Cells Expressing FR-β

FR-β has a K_{d} value of ˜1 nM for folic acid. Folic acid retains a high avidity for FR following covalent derivatization of its γ-carboxyl group. For example, folate-conjugated polyethylene glycol (PEG) has been reported to have an affinity for FR that is only 5-fold lower than that of folic acid. Following binding to FR, folate conjugates have been shown to be internalized by the cell via receptor-mediated endocytosis. Moreover, the intracellular routing of folate conjugates following receptor binding appears to follow a non-degradative pathway.

A variety of folate-conjugated compounds have been developed, primarily for treatment of gynecological tumors, by targeting FR-α. For example, folate has been conjugated to protein toxins, enzymes, antibodies, radiopharmaceuticals, antisense deoxyribonucleotides, chemotherapy agents, starburst dendrimers, and gene transfer vectors. In addition, folate-conjugates may contain other cytotoxic drugs, polymers, nanoparticles, micelles, other biocompatible materials and the like.

Any of the above folate-conjugated therapeutics may be used in the present invention. Such therapeutics have in common that they contain folate that can bind to FR on cells to which the therapeutic is to be targeted. Such therapeutics also have in common that they contain one or more therapeutic substances that are brought into proximity with the cell to which the folate binds such that it can act on the cell. The preferred therapeutic substance is a cellular cytotoxic agent that, when brought into proximity with a leukemia cell, is able to kill the cell.

The preferred folate-conjugated therapeutic in this invention are folate-coated liposomes. Because of their capacity to carry a large payload of drugs, prolonged systemic circulation time, and ability to bind FR with high affinity via multivalent interaction, folate-coated liposomes are especially well-suited for use in the present method. Attachment of folate to the liposome is such that the folate is available to interact or bind with FR, specifically FR-β, on the surface of leukemia cells. The folate-coated liposomes also have one or more therapeutic substances associated with or contained within it.

It should be noted that alternatives to liposomes can be used in the present invention. For example, a variety of particles made of biocompatible materials can also be used. Such particles generally are between 0.1 and 5 microns in diameter. The particles are preferably biodegradable. The particles contain or have attached to them, therapeutic substances. The particles are attached to folate either directly or through another molecule, such as the hydrophilic molecules (e.g., polyethylene glycol or PEG) described below.

Liposomes

A variety of different types of liposomes are well known in the art. Generally, liposomes are spherical particles containing an internal cavity. The walls of liposomes generally are comprised of a bilayer of lipids, particularly phospholipids. There are numerous lipids and phospholipids that can be used to make liposomes. These lipids and phospholipids are well known in the art. Many of the types of liposomes, as well as the lipids and phospholipids, are described in references such as U.S. Pat. No. 5,049,389, and Storm and Crommelin, 1998, Pharmaceutical Science and Technology Today, 1:19-31, the descriptions within these references being herein incorporated by reference.

The liposomes of the present invention may be prepared by any of the standard methods known in the art for making liposomes. A variety of such methods are known. Some such methods include hydration of dried lipids, introduction of an organic solution of lipids into an aqueous solution followed by evaporation of the organic solution, and dialysis of an aqueous solution of lipids and detergents or surfactants to remove the detergents or surfactants. Many of these methods are described in U.S. Pat. No. 5,049,389, Storm and Crommelin, 1998, Pharmaceutical Science and Technology Today, 1:19-31, and Szoka and Papahadjopoulos, 1978, Proc Natl Acad Sci USA, 75:4194-8, the descriptions of which are incorporated herein in their entirety by reference.

Polyethyleneglycol or other hydrophilic polymer-derivatized lipids can be incorporated (usually 1-20 mole %) to provide prolonged systemic circulation. Cholesterol can be incorporated up to 50 mole % to improve bilayer stability. The liposomes may be frozen and/or lyophilized in the presence of a cryoprotectant, such as glucose, sucrose, galactose, etc. and reconstituted at a later date. This will extend the shelf-life of the liposomes.

For systemic drug delivery, stable large unilamellar vesicles (LUVs) with diameters in the range of 100-200 mn, composed of high phase transition temperature synthetic lipids and cholesterol are often used due to their superior stability in circulation. A typical liposome preparation has the basic lipid composition of distearoylphosphatidylcholine/cholesterol (DSPC/Chol) at 60:40 (mol/mol). In addition, a PEGylated lipid mPEG2000-DSPE may be incorporated into the bilayer at 4-10 mole % to sterically stabilize the liposomes against serum protein opsonization, which results in the rapid removal of the liposomes by mononuclear phagocytic cells of the reticuloendothelial system (RES). This prolongs the systemic circulation time of liposomes and reduces their uptake by the liver and the spleen.

After liposomes are made, there are techniques well known in the art for manipulating the liposomes that can also be used in practice of the present invention. For example, a preparation of liposomes made by standard techniques may vary in size and lamellarity (i.e., wall thickness) after they are made. Techniques such as subjecting the liposomes to a high shearing force, extrusion of the liposomes through membranes, or sonication of the liposomes can be used either to select liposomes of a desired size or modify the liposomes such that they have a desired size. After manipulation of liposomes by these methods, the size distribution of the liposomes can be measured to ensure that liposomes of the desired size have been obtained.

Folate-Coated Liposomes

Folic acid is preferably incorporated into or attached to a liposome by attaching or coupling to a hydrophobic anchor (e.g., a molecule that is part of the lipid bilayer of the liposome). The folate can be directly attached to the hydrophobic anchor. For example, folic acid can be directly linked to the head group of a phosphatidylethanolamine (PE) lipid anchor. Preferably, however, folate is attached to the hydrophobic anchor through another, hydrophilic molecule. A preferred hydrophilic molecule for this purpose is polyethylene glycol (PEG). We have used PEG of molecular weight 3,350 for this purpose, although PEG of other molecular weights can be used. Liposomes that have folate attached in any of these ways are called herein termed “folate-coated liposomes.”

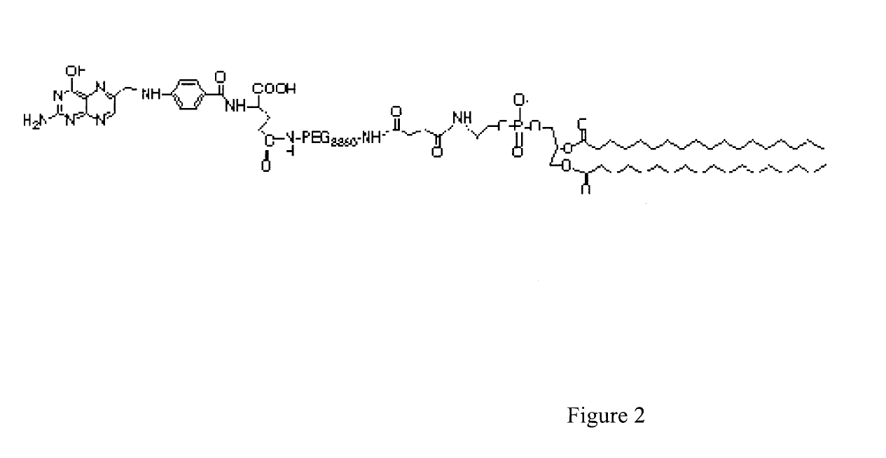

Procedurally, attachment of folate to liposomes can be performed either by attaching folate to preformed liposomes or by attaching folate to the liposomes as the liposomes are made. The latter method is preferred and is preferably accomplished by incorporating into the liposome formulation, a pre-synthesized lipophilic folate derivative (i.e., folate attached to a lipid that becomes part of the lipid bilayer of the liposome). Again, the folate can be directly attached to the lipid or can be indirectly attached, through a molecule such as PEG. One such folate-PEG-lipid molecule is folate-polyethyleneglycol-distearoyl phosphatidylethanolamine (Folate-PEG-DSPE) (FIG. 2). Another such molecule is folate-polyethyleneglycol-cholesterol (Folate-PEG-Chol). Other molecules of similar structure can also be used and can be made using a variety of methods.

Generally, such molecules can be of the chemical composition “folate-linker1-polymer-linker2-hydrophobic anchor.” “Linker1” and “linker2” can be of any type, such as ester, ether, amide, hydrozone, thioether, disulfide, etc. “Polymer” can be of any type, preferably water soluble. “Hydrophobic anchor” can be any fatty alkyl, acyl, or amido derivatives or cholesterol derivatives.

Methods for attaching folate to preformed liposomes can be performed by either covalent derivatization of the liposome surface via reactive functional groups in the lipids, such as an amino group on phosphatidylethanolamines, or by mixing micellar suspensions of the lipophilic folate conjugates with preformed liposomes to allow the conjugates to spontaneously partition into the outer leaflet of the liposomal bilayer.

Methods for attaching folate to liposomes by incorporating a pre-synthesized lipophilic folate derivative into the liposome composition as the liposomes are made can be performed by co-dissolving folate-PEG-DSPE or folate-PEG-Chol with other lipid ingredients in a single or a mixture of organic solvents at the beginning of the standard liposome preparation process, which can include sonication, homogenization, reverse-phase evaporation, high pressure extrusion, organic solvent injection, or detergent solubilzation followed by detergent removal by dialysis. One method for synthesizing such molecules is described in detail in Example 1.

Liposomes Containing Therapeutic Agents

The folate-coated liposomes of the present invention contain therapeutic agents, are attached to therapeutic substances, or are associated with therapeutic substances, such that the substances are delivered to the leukemia cells to which the folate part of the folate-conjugated therapeutic. Such liposomes are said to be “loaded” with therapeutic substances. The methods whereby the liposomes become loaded with therapeutic substances is referred to as “loading.”

Substances delivered using liposomes exhibit prolonged systemic circulation time and reduced peak plasma concentrations compared to those of the free drug (i.e., not delivered using liposomes), which are often subjected to rapid urinary excretion. Therefore, liposomal delivery is associated with a large increase in plasma half-life (t_{1/2}), mean residence time (MRT), and the area under the plasma concentration versus time curve (AUC) of the drug.

There are a variety of methods known in the art for loading therapeutic substances or drugs into liposomes. Generally, drugs which are hydrophilic in nature are located or associated with the internal cavity of the liposome particles. Generally, drugs which are lipophilic in nature are located or associated with the lipid bilayer of liposome particles. In passive loading, liposomes are formed in a solution of the drug to be used. In active loading, a variety of methods are known in the art. In one type of active loading, called pH based loading, a proton is dissociated from the drug molecules, causing the drugs to enter into and remain in the liposomes (Ceh and Lasic, 1997, J Colloid Interface Sci, 185:9-18.). In ammonium sulfate loading, the drug is caused to enter into liposomes due to presence of an ammonium sulfate gradient (Ceh and Lasic, 1997, J Colloid Interface Sci, 185:9-18.). In another method, a chelating agent within the liposome results in trapping of drug therein as well as further diffusion of drug into the liposomes (Patent No. WO0023052).

After the loading of drug into the liposomes, steps may be used to remove drug that has not been loaded and is not associated with the liposomes. Such steps may comprise techniques such as ion exchange, diafiltration, or washing of the particles or agglomerates using ultracentrifugation.

A variety of therapeutic agents or drugs can be used in the folated-coated liposomes of the present invention. Doxorubicin, donarubicin, vincristine and many other agents, alone or in combination, can be used. Preferably, the drugs are cytotoxic to cells to which they are brought in close contact with.

A preferred chemotherapeutic agent is doxorubicin (DOX). DOX can be quantitatively loaded into preformed liposomes via a remote-loading procedure, which is based on a transmembrane pH-gradient. Other agents can be loaded into liposomes using methods well known in the art.

Pharmaceutical Compositions

The FR-β inducers and folate-conjugated therapeutics are preferably parts of pharmaceutical compositions intended for administration to a patient. Preferably, separate pharmacuetical compositions contain FR-β inducers and folate-conjugated therapeutics, however, a single pharmaceutical composition may contain both an FR-β inducer and folate-conjugated therapeutic. The particular pharmaceutical composition will depend on the method by which the composition is administered to a patient. However, pharmaceutical compositions routinely comprise salt, buffering agents, preservatives, other vehicles and, optionally, other therapeutic agents in addition to the FR-β inducers and/or folate-conjugated therapeutics contained therein.

Compositions suitable for parenteral administration are preferred and conveniently comprise a sterile, pyurogen-free, aqueous or oleaginous preparation of FR-β inducers and/or folate-conjugated therapeutic, which is preferably isotonic with the blood of the recipient. This aqueous preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride. solution. In addition, sterile, fixed oils are conventionally employed as a solvent or suspending medium. For this purpose any bland fixed oil may be employed including synthetic mono- or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, Pa. The pharmaceutical compositions may conveniently be presented in unit dosage form and may be prepared by any of the methods well-known in the art of pharmacy.

Additionally, preparation of parenterally-acceptable solutions of the pharmaceutical composition, having due regard to pH, isotonicity, stability, and the like, is within the level of ordinary skill in the art of pharmacology. A preferred pharmaceutical composition for injection can contain, in addition to the vector, an isotonic vehicle such as Sodium Chloride Injection, Ringer's Injection, Dextrose Injection, Dextrose and Sodium Chloride Injection, Lactated Ringer's Injection, phosphate buffered saline (PBS), or other vehicle as known in the art. The pharmaceutical composition used in the method of the present invention may also contain stabilizers, preservatives, buffers, antioxidants, or other additives known to those of skill in the art.

Administration of the Pharmaceutical Compositions

The invention comprises administration of one or more FR-β inducers and one or more folate-conjugated therapeutics, to a patient, preferably as part of one or more pharmaceutical compositions.

The patients to whom the treatment is given will generally be those who have leukemia. Preferably, the treatment is given to a patient known to have AML. Diagnosis of leukemia in general and AML in particular is known in the art of medicine and oncology and will normally be performed by one or more physicians.

Generally, the FR-β inducers are administered to the patient at a point in time before the folate-conjugated therapeutic is administered to the patient, although it is possible to administer the FR-β inducers and folate-conjugated therapeutic at the same time to a patient. Preferably, the FR-β inducers are administered first and the folate-conjugated therapeutic is administered at a time thereafter when expression of FR-β on the leukemia cells of the patient are at a maximum.

The pharmaceutical compositions, generally speaking, may be administered using any mode that is medically acceptable, meaning any mode that produces the desired increase in FR-β receptors and/or anti-leukemia activity, without causing clinically unacceptable adverse effects. The pharmaceutical compositions can be administered locally or systemically, using routes of administration described below. Such modes of administration include parenteral routes (e.g., intravenous, subcutaneous, intramuscular, intraperitoneal, mucosal or infusion), but may also include oral, rectal, topical, nasal or intradermal routes. Other delivery systems can include time-release, delayed release or sustained release delivery systems. Such systems can avoid repeated administrations, increasing convenience to the subject and the physician. Many types of release delivery systems are available and known to those of ordinary skill in the art. The pharmaceutical compositions are administered once or repeatedly in a therapeutically or biologically effective amount.

Those skilled in the art will recognize that delivery via injection contemplates the use of a syringe, catheter or similar device, which delivers the pharmaceutical composition to a site. Delivery, preferably, results in the FR-β inducers and folate-conjugated therapeutic being systemically distributed throughout the circulatory system of the patient.

Although the drugs administered by the folate-conjugated method may be cytotoxic drugs, other drugs can also be administered in this way. Such drugs may include diagnostic imaging agents, pro-drug converting enzymes, immuno-modulating agents, cytokines, growth factors, antibodies, agonists, antagonists, and other drugs.

The amount of FR-β inducer and folate-conjugated therapeutic administered to the patient is described as a biologically effective amount. As used herein, the term “biologically effective amount” means the total amount of each active component of the pharmaceutical formulation or method that is sufficient to show a meaningful subject or patient benefit, i.e., an increase in expression of FR-β on leukemia cells in the case of the FR-β inducer.

A biologically effective amount of folate-conjugated drug will depend upon the nature and severity of the leukemia being treated, and on the nature of prior treatments which the patient has undergone. Initially, the attending physician may administer low doses of the composition and observe the patient's response. Larger doses of composition may be administered until the optimal therapeutic effect is obtained for the patient, and at that point the dosage is not increased further. It may be desirable to administer simultaneously or sequentially a therapeutically effective amount of one or more of the therapeutic compositions of the invention to one patient as a single treatment episode. Ultimately, the attending physician will decide the amount of therapeutic composition with which to treat each individual patient.

A biologically effective amount of the folate-conjugated therapeutic will preferably result in elimination of leukemia cells from the patient's body. Preferably, as a result of the inventive treatment, the patient will go into remission from the leukemia, will stay in remission and will become a long-term survivor, preferably dying from some affliction other than the leukemia or the inventive treatment. However, it is not necessary that remission and long-term survival be the result of the inventive treatment. Any increase in the lifespan of the patient with the inventive treatment as compared to lifespan without the inventive treatment is a desirable goal. Alternatively, effects of the inventive treatment may be measured as an improvement in the quality of life of the patient or patient suffering, absent an increase in lifespan of the patient.

The duration of therapy with the pharmaceutical compositions used in the method of the present invention will vary, depending on the unique characteristics of the pharmaceutical composition and the particular therapeutic effect to be achieved, the severity of the disease being treated and the condition and potential idiosyncratic response of each individual patient. Ultimately the attending physician will decide on the appropriate duration of therapy with the pharmaceutical composition used in the method of the present invention.

Although dietary restrictions should not be required, the patients may be advised not to consume vitamin tablets containing folate in the period prior to and for the duration of treatment with FR-targeted therapy, since it is conceivable that taking these tablets could result in transient elevation of serum folate to levels higher than normal. Since folate is a low molecular weight vitamin with very rapid renal clearance, any dietary effect on serum folate would be for the short term.

### EXAMPLES

The invention may be better understood by reference to the following examples, which serve to illustrate but not to limit the present invention.

### Example 1

Preparation and Testing of a Folate-Conjulated Liposome Therapeutic

Preparation of Folate-Coated Liposomes

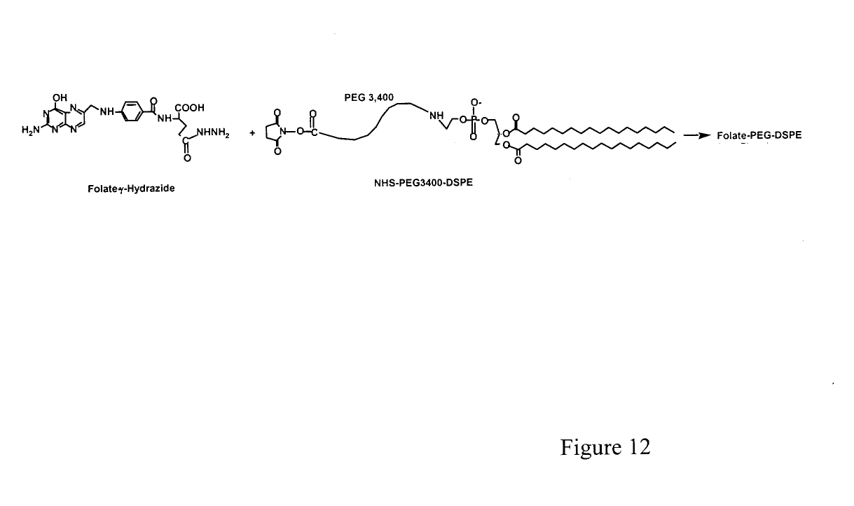

Folate (γ)-PEG-DSPE was synthesized as follows (FIG. 12): (i) folate-γ-hydrazide was synthesized by reacting an activated NHS ester of folate with hydrazine hydrate (see Guo, Hinkle and Lee, 1999, J. Nucl. Med., 40:1563-1569); (ii) folate-PEG-DSPE was synthesized by reacting folate-γ-hydrazide with NHS-PEG3400-DSPE (Sheerwater Polymers, Inc.); and (iii) the final product (Folate-PEG-DSPE; FIG. 2) was purified on a reverse-phase preparative HPLC using an established solvent system.

Folate-coated liposomes containing DOX (f-L-DOX) were made as described below. Liposome preparation was performed by polycarbonate membrane (with 100-nm pore size) extrusion at 60° C. using a N2 driven high pressure Lipex™ extruder (also see Example 3). A composition of DSPE/Chol/mPEG-DSPE/folate-PEG-DSPE (60:36:4:0.1, mole/mole) was used. All lipid components were purchased from Avanti Polar Lipids. DOX was loaded into liposomes by remote-loading driven by a transmembrane pH-gradient, at a drug to lipid ratio of 1:10 (w/w). Liposomal DOX was purified by gel-filtration on a Sepharose CL-4B column and sterilized by filtration through a 0.2-μm cellulose acetate syringe filter. The loading efficiency was determined by comparing the drug-to-lipid ratio of the product versus that of the starting materials, and was found to be >95%. DOX concentration in the liposome preparation was determined by dissolving the L-DOX in methanol and measuring absorbance at 480 nm based on the known extinction coefficient of DOX. Lipid concentration was determined by an ammonium ferrithiocyanate calorimetric assay, or by liquid scintillation counting for liposomes labeled with a trace amount of [³H] cholesterol hexadecylether (Amersham). Liposome size distribution was determined by photon correlation spectroscopy on a Nicomp 370 laser submicron particle sizer and showed a mean diameter of ˜100 nm. Stability of the liposomal doxorubicin upon storage in PBS or in 50% human serum was determined by re-purifying the liposomes by gel filtration and determination of the drug-to-lipid ratio. Significant decreases in this ratio would indicate DOX release from the liposomes. Leakage was <1%.

Uptake of Folate-Coated Liposomes by FR-β Expressing Cells

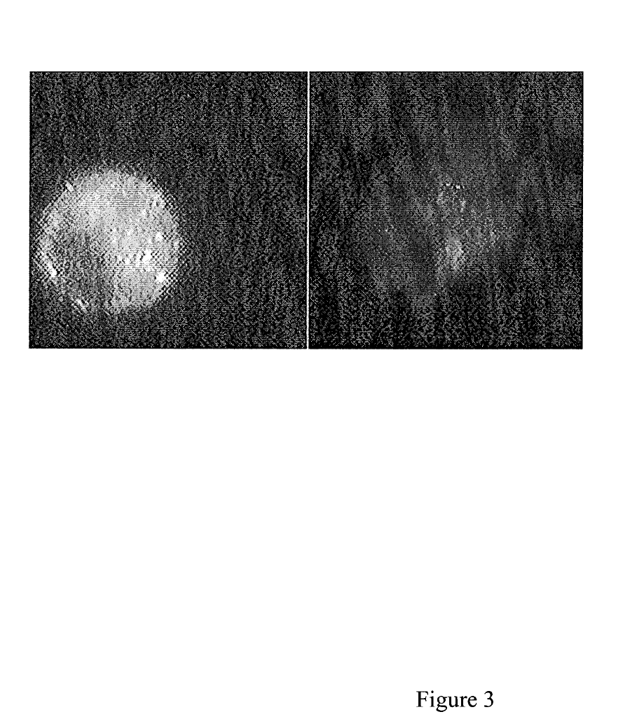

Folate-coated liposomes with the composition DSPC/Chol/folate-PEG-DSPE (60:40:0.1, m/m) and entrapping calcein, a membrane impermeable fluorescent dye, (f-L-calcein) were prepared, using a modification of the procedure described above. Non-targeted calcein-containing liposomes (L-calcein), with essentially the same composition, minus folate-PEG-DSPE, were used as a control. FR (+) cell lines, human KG-1, CHO-FR-β, and murine L1210JF, and the FR (−) cell lines KG-1a, CHO-K1 and L1210 were used. FR-β expression on the cells was determined by flow cytometry using a receptor-specific antibody. For liposome binding studies, the cells were treated in triplicate with f-L-calcein (folate-conjugated liposomes containing calcein) or free L-calcein at 20 μM in media. The cells were then washed 3× with cold PBS and examined by fluorescence microscopy (FIG. 3). The data showed that the overall cellular fluorescence was much greater in cells treated with f-L-calcein compared to cells treated with free L-calcein. These data indicated that f-L-calcein was internalized by the cells.

Targeting of Compound Contained in Folate-Coated Liposomes to Cultured Cells with Enhanced Levels of FR-β on the Plasma Membrane

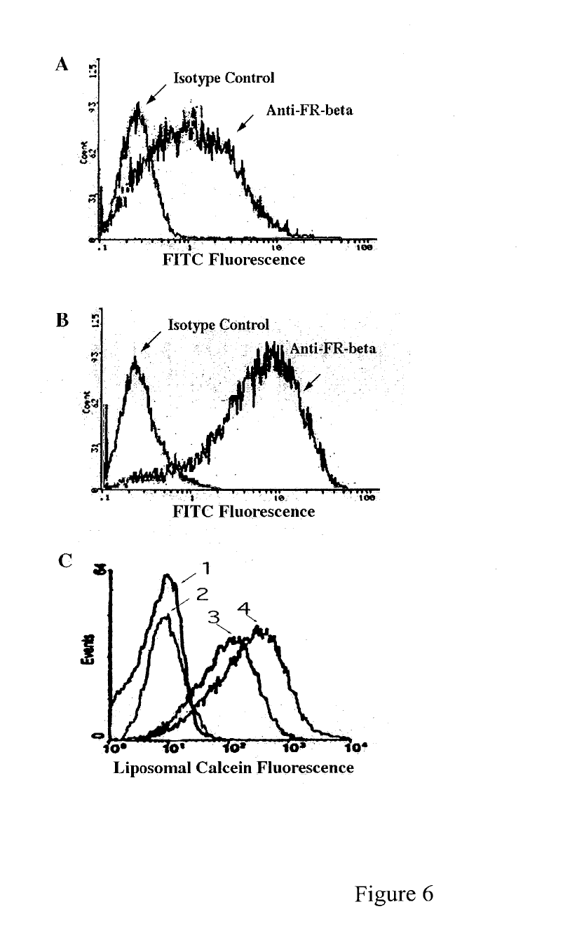

Uptake of folate-coated liposomes by cells with or without FR-β, in the absence or presence of ATRA, was studied. Uptake of calcein, contained in the liposomes, was measured by flow cytometry. As shown in FIG. 6, treatment of KG-1 cells with ATRA resulted in a significant increase in FR-β expression (Panels A and B). ATRA treatment also resulted in a significant increase in targeted calcein uptake (Panel C). Data on cellular uptake of calcein-containing liposomes in the FR (+) KG-1, L1210JF and CHO-FR-β cells, and FR (−) KG-1a, L1210 and CHO-K1 controls are summarized in Table 2 below. The data in Table 2 represent the relative fluorescence index (fluorescence intensity) of the cell populations.

TABLE 2
Cellular uptake of f-L-calcein by various cell lines determined by flow cytometry
(n = 3).

| | FR(+) | | | | FR(−) | | | |
|---|---|---|---|---|---|---|---|---|
| Cell Line | KG-1 | | L1210JF | CHO-FR-β | KG-1a | | L1210 | CHO-K1 |
| Pre-exposure to ATRA | − | + | − | − | − | + | − | − |
| L-calcein | 9.3 ± 2.1* | 9.1 ± 1.3 | 5.7 ± 0.6 | 6.1 ± 1.3 | 5.4 ± 1.3 | 5.1 ± 0.3 | 4.7 ± 0.3 | 5.1 ± 2.3 |
| f-L-calcein | 61 ± 17 | 320 ±78 | 78 ± 9 | 91 ± 12 | 6.1 ±0.7 | 6.7 ± 0.9 | 7.1 ± 1.2 | 5.7 ± 1.5 |
| f-L-calcein + | 11 ± 1.4 | 27 ± 2 | 8.3 ± 1.7 | 7.3 ± 0.2 | 5.1 ± 0.5 | 7.3 ± 1.4 | 6.4 ± 1.5 | 4.9 ± 0.7 |
| 1 mM folate | | | | | | | | |
| f-L-calcein + | 63 ± 11 | n/d** | 81 ± 13 | n/d | n/d | n/d | n/d | n/d |
| 1 μM folate | | | | | | | | |
| f-L-calcein + | 13 ± 4 | n/d | n/d | n/d | n/d | n/d | n/d | n/d |
| pretreated w/ PI-PLC | | | | | | | | |
| f-L-calcein + | 16 ± 6 | n/d | n/d | n/d | n/d | n/d | n/d | n/d |
| antiserum for FR-β | | | | | | | | |

In all cases, substantially greater cellular uptake of f-L-calcein was observed compared to non-targeted liposomes when FR-β was expressed. KG-1 cells treated with f-L-calcein had a 36-fold higher mean fluorescence intensity than those treated with non-targeted L-calcein. Moreover, f-L-calcein uptake was increased by an additional 5-fold when pre-treated with 1 μM ATRA, which could be correlated with the increase in FR-β expression in response to ATRA. Furthermore, uptake of f-L-calcein in KG-1 cells was blocked by 1 mM free folic acid, pre-treatment of the cells with phosphatidylinositol-specific phospholipase C (PI-PLC) which releases cell surface FR-β by cleaving its GPI membrane anchor, or pre-incubation of the cells with antiserum against FR-β indicating that FR-β was responsible for cellular uptake of f-L-calcein. In contrast, the FR (−) KG-1a subline showed neither FR-β expression nor preferential uptake of f-L-calcein with or without ATRA pre-treatment. These data showed that FR-β in cultured cells mediated the selective uptake of folate-coated liposomes.

In order to examine the potential effect of physiological folate on FR-mediated drug delivery in FR-β (+) cells, f-L-calcein uptake was measured in KG-1 cells in the presence of 0-10 μM 6S-5-methyltetrahydrofolate (6S-5MeTHF) using the same flow cytometry method as described above. As shown in Table 3, even at 10 μM (>200× median human physiological serum folate levels), 6S-5MeTHF did not significantly inhibit f-L-calcein uptake.

TABLE 3
Effect of 6S-5-MeTHF on f-L-calcein uptake in KG-1 cells.

| | | | | |
|---|---|---|---|---|
| 6S-5MeTHF Conc. | 0 | 0.1 μM | 1 μM | 10 μM |
| f-L-calcein uptake | 59 ± 15 | 57 ± 18 | 58 ± 13 | 53 ± 14 |

In addition, normal human serum folate is in the form of 6S-5MeTHF, for which FR-β has ˜35× lower affinity than folic acid. Therefore, serum folate, even at the high end of the physiological range, does not pose a significant obstacle for the efficient targeting of FR-β-bearing cells via folate-conjugated liposomes.

Cytotoxicity of f-L-Dox to Cultured Leukemia Cells

FR-targeted liposomal doxorubicin (f-L-DOX) was evaluated for in vitro cytotoxicity in both FR (+) and FR (−) leukemia cells using a standard cell survival assay (MTT assay). FR-targeted liposomal-doxorubicin (f-L-DOX) was prepared as described above. F-L-DOX in vitro cytotoxicity was evaluated in two FR (+) cell lines [KG-1, an FR-β (+) human AML cell line, and L1210JF, an FR (+) subline of L1210 murine acute lymphocytic leukemia cell line] and two FR (−) cell lines [KG-1a, an FR-β (−) subline of KG-1, and FR (−) L1210 cells] using the survival assay. The cells were treated in quadruplicate with serial dilutions of f-L-DOX, non-targeted control L-DOX, or free DOX. Cell viability was determined and IC₅₀ values were calculated. For KG-1 and KG-1a cells, the MTT assay was repeated with cells cultured in media containing 1 μM ATRA for 5-days.

The IC₅₀ values (Table 4) show that f-L-DOX was 25 times more cytotoxic than L-DOX to the FR-β (+) KG-1 cells in the absence of ATRA, and 63 times more cytotoxic with ATRA pre-treatment. In contrast, no therapeutic advantage or an ATRA-induction effect on cytotoxicity was observed with f-L-DOX in the KG-1a cell line, which is FR-β (−). Superior cytotoxicity of f-L-DOX over L-DOX was also observed in the FR (+) L1210JF cell line, but not in the FR (−) L1210 cells. Data in Table 4 further showed that free folate (1 mM) increased the IC₅₀ of f-L-DOX in KG-1 cells (ATRA treated and untreated), and L1210JF cells by about an order of magnitude.

TABLE 4
Cytotoxicity of folate-L-DOX to Cultured Leukemia cells

| | Leukemia Cell Line | | | | | |
|---|---|---|---|---|---|---|
| | KG-1 | | KG-1a | | L1210JF | L1210 |
| | FR expression status | | | | | |
| | FR-β (+) | | FR (−) | | FR-α (+) | FR (−) |
| Pre-exposure to ATRA | − | + | − | + | − | − |

a. IC₅₀ values (μM) for cells incubated in folate-free media

| | | | | | | |
|---|---|---|---|---|---|---|
| Free DOX | 0.65 | 0.63 | 0.61 | 0.59 | 0.86 | 0.91 |
| Folate-L-DOX | 0.97 | 0.43 | 19 | 17 | 1.3 | 27 |
| Non-targeted L-DOX | 24 | 27 | 23 | 22 | 34 | 29 |

b. IC₅₀ (μM) values for cells incubated in media containing 1 mM
folic acid

| | | | | | | |
|---|---|---|---|---|---|---|
| Free DOX | 0.67 | 0.68 | 0.63 | 0.62 | 0.87 | 0.93 |
| Folate-L-DOX | 11 | 5.4 | 21 | 19 | 12 | 29 |
| Non-targeted L-DOX | 27 | 29 | 23 | 20 | 35 | 31 |

The assays were repeated twice. Within each assay, the cells were treated in quadruplicate. The error within each group was <20%.

### Example 2

Properties of a Radiolabeled Folate Conjugate, ^{99m}Tc-HYNIC-folate

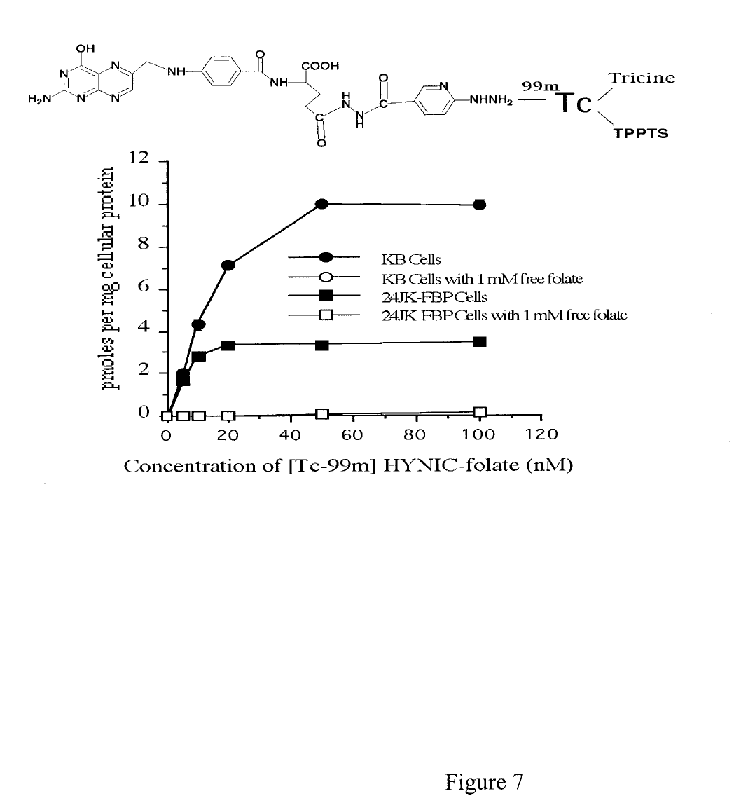

To evaluate the properties of folate conjugates, a radiolabeled folate conjugate, hydrazinonicotinamide-folate (HYNIC-folate) was synthesized and labeled with γ-emitting radionuclide ^{99m}Tc using tricine and triphenylphosphine (TPPS) as co-ligands (FIG. 7).

In one study, the [^{99m}Tc]HYNIC-folate was added to the medium of cultured cells. The cells were then washed to remove unbound [^{99m}Tc]HYNIC-folate, and then counted in a γ-counter. The results (FIG. 7) showed that the radioconjugate exhibited high specificity for FR-β-mediated uptake in cultured FR-β (+) KB oral carcinoma cells and FR-β transfected murine sarcoma 24JK-FBP cells (obtained as a gift from Dr. Patrick Hwu at the NCI) (FIG. 7). The binding of the radioligand in both cell lines was saturable and was completely inhibited by 1 mM free folate (FIG. 7).

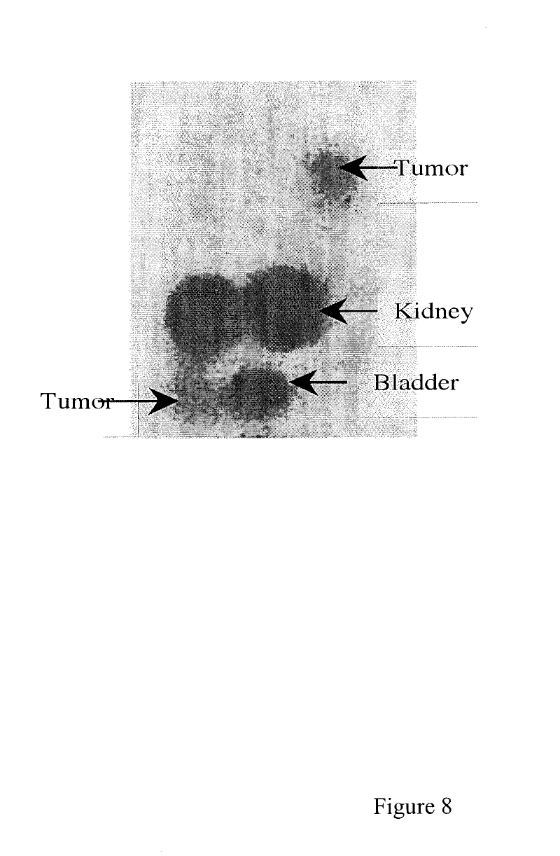

A biodistribution study was then carried out in mice carrying implanted syngeneic 24JK-FBP subcutaneous tumors. Seven to eight week-old female C57BL/6 mice on a folate-deficient rodent diet were inoculated subcutaneously at two separate sites (right shoulder and left hip) with ˜2×10⁶ 24JK-FBP cells 2 weeks before the study. During the study, mice were injected with 100 μL of ^{99m}Tc-HYNIC-folate via the lateral tail vein and sacrificed at either 4 hours or 24 hours post-injection. Gamma-camera images (FIG. 8) of the animals at the 4 hour time point were taken immediately prior to sacrifice. Tissue samples were harvested, weighed and counted in an automatic well γ-counter. Tumor-to-background tissue ratios of 55 to 1 and 81 to 1 were obtained for the radioactive signal at the 4 hour and 24 hour time points, respectively.

The only normal tissue that showed significant radiotracer uptake was the kidney (FIG. 8). Radiotracer uptake in the kidneys and tumor were 61% and 17% injected dose/gram tissue (% ID/g), respectively. Tumor uptake of the radioconjugate was 90% blocked by co-injection of 100 μg free folate. The high level of radioactivity found in the kidney is due to the presence of FR in the apical proximal tubules since kidney uptake was also blocked by the free folate co-injection. Approximately 50% of the radioactivity in the kidneys and 30% in the tumors remained at the 24 hour time point, indicating that a significant fraction of the folate conjugate was internalized by the tumor and kidney cells.

The result of the biodistribution study shows that the folate radioconjugate was not taken up by normal tissues, except for the kidneys. In another study, a ¹²⁵I-BSA-folate radioconjugate did not show increased kidney uptake as compred to non-conjugated BSA. In addition, biodistribution studies of folate-conjugated liposomes did not show uptake by the kidney.

Taken together, these results indicate that low molecular weight folate conjugates readily pass through the glomerular membrane and accumulate at high levels in the kidneys via FR-β-mediated resorption. However, high molecular weight drugs or drug carriers, such as folate-conjugated liposomes, are not subject to glomerular filtration due to their size and, therefore, do not accumulate in the kidneys.

### Example 3

FR Binding to Cultured Cells and Tumor Localizing Properties in Experimental Animals of Radiolabeled Folate-Coated Liposomes

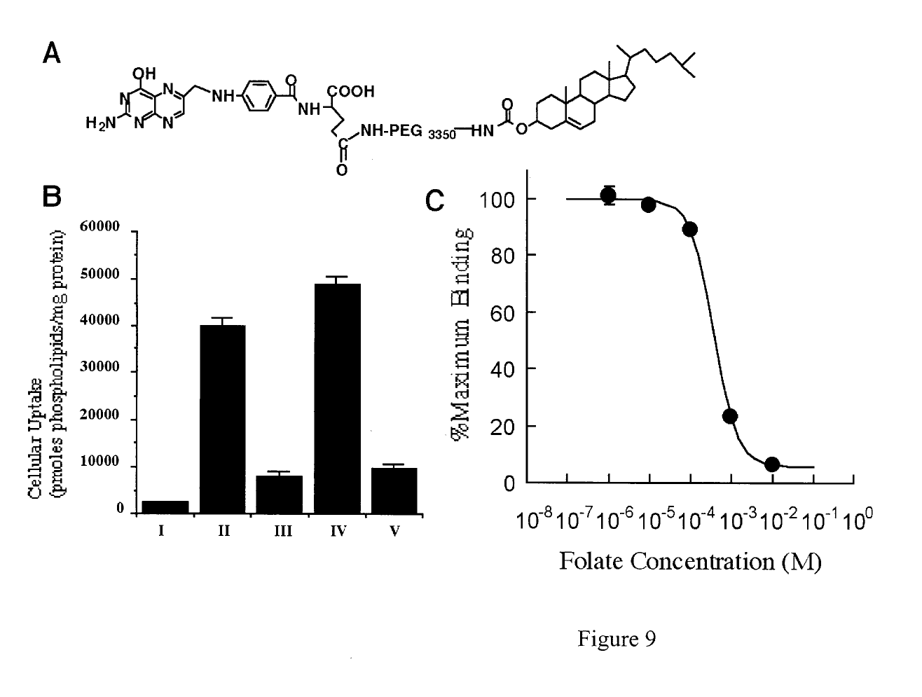

A novel folate-derivative, folate-PEG-Chol (FIG. 9A), was synthesized by reacting folate-PEG (MW˜3,350)-amine with cholesterol chloroformate. The folate-derivative was then incorporated into liposomes. Lipid compositions for folate-coated and non-targeted control liposomes were DSPC/Chol/mPEG2000-DSPE/folate-PEG-Chol (60/34/5/1, m/m) and DSPC/Chol/mPEG2000-DSPE (60/34/6, m/m), respectively. An additional 1 mole % of DTPA-DSPE was added into the formulation to allow for liposome radiolabeling with ¹¹¹In. The liposomes were prepared by high-pressure extrusion using an Avestin hand-held extruder with a 100-nm pore-size polycarbonate membrane. Liposome mean diameter was measured by photon-correlation spectroscopy on a Nicomp 370 submicron particle size analyzer and found to be 104 nm. The liposomes were radiolabeled by incubating with ¹¹¹InCl in a citrate buffer and were purified by size-exclusion chromatography on a Sepharose CL-4B column.

The liposomes were added to cells. Uptake of liposomes containing folate-PEG-Chol in KB cells was comparable to that of liposomes containing folate-PEG-DSPE and ˜20-fold higher than that of the non-targeted control liposomes (FIG. 9B). The cellular uptake of the FR-targeted liposomes was competitively blocked by free folate. The apparent K_{d} for FR-targeted liposomes, based on a competitive binding assay (FIG. 9C) and using the equation Kᵢ=IC₅₀/(1+[liposomes]/_{Kd-app}), was calculated to be 1.5×10⁻¹⁵ M. The affinity of folate-coated liposomes for FR (+) cells was 10⁵ times higher than the affinity of folic acid alone.

A biodistribution study was conducted in the C57BL/6 murine tumor model generated by subcutaneous engraftment of the FR (+) 24JK-FBP cells, which is desribed above in Example 2. The animals were injected intravenously with ¹¹¹In-labeled liposomes containing 50 μg of phospholipids and ˜25 μCi of radioactivity, with or without intraperitoneal injection of 1 mg of free folic acid. The animals were sacrificed at 24 hours and the tissue samples analyzed for radioactivity. Even though the tumor versus surrounding normal tissue uptake of the folate-derivatized liposomes was high (60:1 for the tumor to muscle ratio), no significant difference in overall tumor localization was observed between the folate conjugated and non-targeted control liposomes, which were 6.5% versus 7.1% ID/g, respectively. The results sugsuggested that, in both cases, the liposomal uptake by the tumor was likely determined primarily by an effect called EPR, for enhanced permeability of the tumor endothelium and the increased drug retention due to lack of lymphatic drainage, which describes a nonspecific accumulation of liposomes in tumors (Yuan, Leunig, Huang, Berk, Papahadjopoulos, and Jain, 1994, Cancer Res. 54:3352-3356, and Hobbs, Monsky, Yuan, Roberts, Griffith, Torchilin and Jain, 1998, Proc. Natl. Acad. Sci. USA, 95:4607-4612). This EPR effect, although observed in solid tumors, is not likely to occur in leukemia, the disease treated with the present invention.

### Example 4

Therapeutic Efficacy of f-L-DOX, L-DOX and free DOX in Murine Leukemia Models

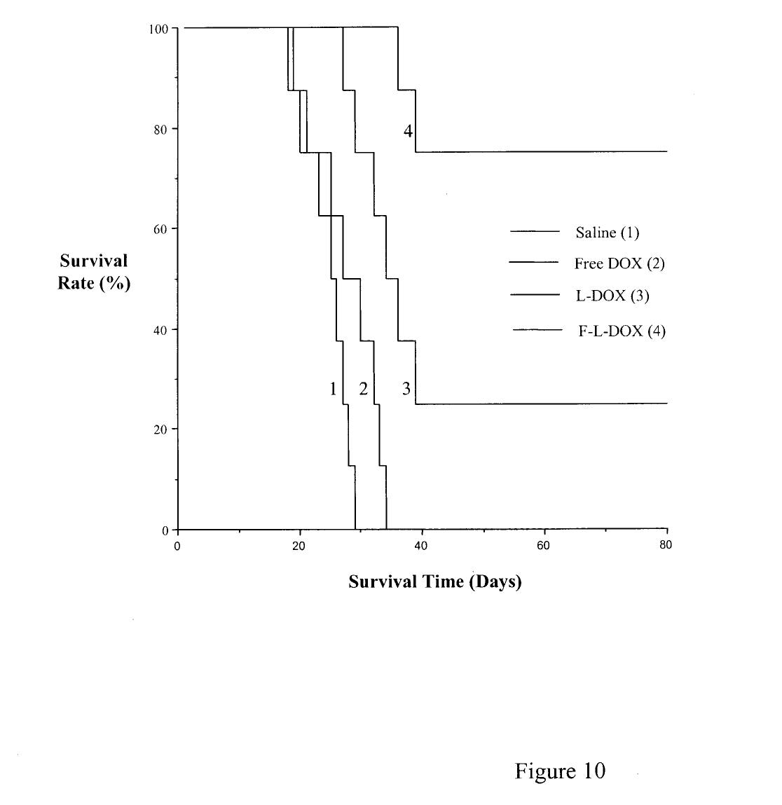

The antileukemic therapeutic efficacy of f-L-DOX was evaluated in two FR (+) murine leukemia ascites tumor models. The first model consisted of DBA/2J mice with ascites tumors derived from intraperitoneally engrafted FR (+) murine lymphocytic leukemia L1210JF cells. Male mice (18-22 g, from Charles River, Wilmington, Mass.) were placed on a folate-deficient diet (AIN-93G from Dyets, Inc., PA) and for at least one week prior to leukemia cell inoculation. The mice, in groups of 8 animals, were injected intraperitoneally with 1×10⁶ L1210JF cells on day 0. The animals then received three intraperitoneal injections (in 50 μL) of various drug formulations on days 1, 5, and 9. The treatment groups were: 1) saline (0.9% NaCl, USP), 2) free DOX (3 mg/kg), 3) L-DOX (5 mg/kg DOX), and 4) f-L-DOX (5 mg/kg DOX). The body weight and survival of the animals were monitored daily. In the saline-treated group, no visible ascitic fluid began to develop until after one week. The results are shown in FIG. 10.

The median survival time for the four groups of mice was 25.5, 28.5, 35 and >80 days, respectively (FIG. 10). The data clearly indicated that L-DOX was more effective than free DOX in the treatment of the FR (+) L1210JF cells and in prolonging median survival time (p-value from log rank test=0.0159), presumably due to the favorable pharmacokinetic properties of L-DOX compared to free DOX. Meanwhile, f-L-DOX was even more efficacious in increasing median survival than L-DOX (p-value from log rank test=0.0259), and increased the long term survival rate to 75%, due to the FR-mediated tumor cell targeting.

The therapeutic efficacy of f-L-DOX in a human leukemia murine ascites xenograft model was further tested. Female CB.17 SCID (scid/scid, 18-22 g) mice were inoculated intraperitoneally with human AML KG-1 cells, which express FR-β, to generate ascites tumors. KG-1 cells (1×10⁶) grown in folate-deficient RPMI1640 media supplemented with 20% FBS were injected into the mice intraperitoneally on day 0. Without treatment, visible ascitic fluid developed at ˜day 30. Peritoneal exudate cells were collected by peritoneal lavage with 5 mL Hanks' balanced salt solution (HBSS), pelleted and resuspended in RPMI1640 media. These cells exhibited a morphology (large size, high nuclear/cytoplasm ratio, fine chromatin structure with visible nucleoli, basophilic cytoplasm) similar to that of KG-1 cells maintained in vitro. Necropsy of the mice showed enlarged spleens and scattered tumor nodules in the peritoneum with no apparent infiltration to the peripheral blood and the bone marrow. The tumor cells were inoculated into 8 groups of 8 mice. Four of the groups received daily intraperitoneal injections of 10 mg/kg of ATRA on days 1 through 5. The following formulations were administered as intraperitoneal injections on days 1, 5, and 9 (in 50 μL): 1) saline, 2) free DOX (3 mg/kg), 3) L-DOX (5 mg/kg), 4) f-L-DOX (5 mg/kg), 5) saline+ATRA, 6) free DOX+ATRA, 7) L-DOX+ATRA, and 8) f-L-DOX+ATRA. The body weight and survival of the animals were monitored daily during the course of the study.

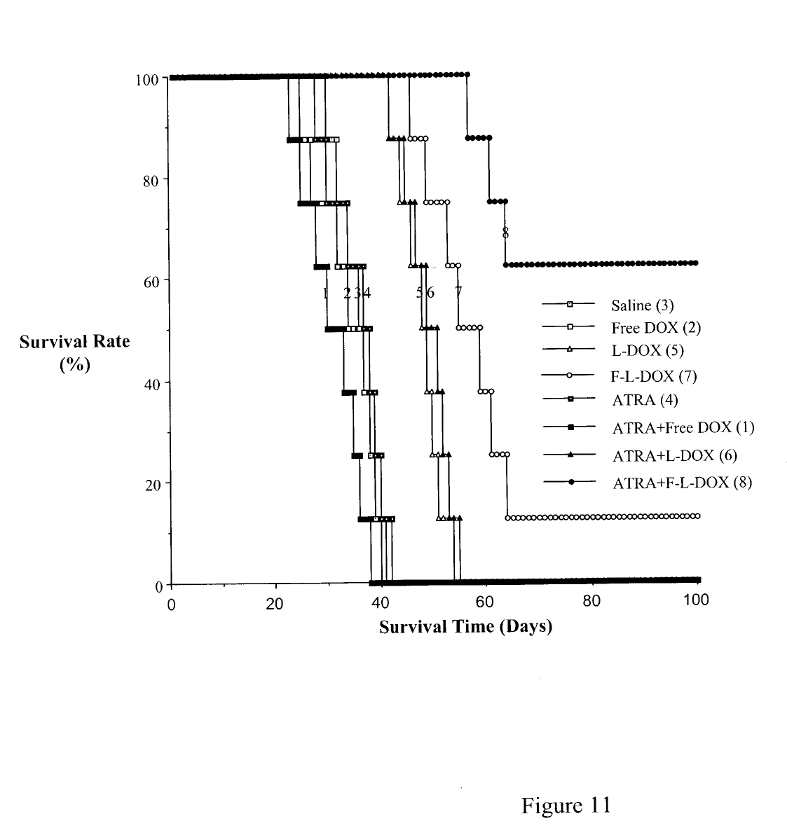

The data showed that L-DOX was more effective than free DOX in extending median survival (p-value from log rank test<0.0001), but did not yield long-term survival (FIG. 11). F-L-DOX was found to be more effective than L-DOX (p-value from log rank test=0.0061), resulting in improved therapeutic efficacy and 12.5% long term survival. ATRA co-treatment further improved the long-term survival rate of the f-L-DOX treated animals from 12.5% to 60% (p-value from log rank test=0.0190). The pronounced improvement in therapeutic efficacy with ATRA co-treatment is likely due to upregulation of FR-β expression on KG-1 cells in vivo, since ATRA does not induce significant differentiation or growth inhibition of KG-1 cells, and ATRA treatment in the absence of f-L-DOX or the L-DOX control did not enhance survival (FIG. 11). Free DOX showed dose-limiting toxicity at >3 mg/kg (results not shown). These results show the validity of the method of enhancing the efficacy of FR-β targeted therapy via receptor upregulation using ATRA.

We claim:

1. A method for treating leukemia in a patient, comprising:
(a) administering at least one FR-β inducer to the patient to increase the levels of FR-β on the leukemia cells in the patient; and
(b) administering a biologically effective amount of a folate-conjugated therapeutic to the patient.

2. The method of claim 1 wherein the leukemia is acute myelogenous leukemia (AML) or chronic myelogenous leukemia (CML).

3. The method of claim 1 wherein FR-β inducer and folate-conjugated therapeutic is administered intravenously or intramuscularly.

4. The method of claim 1 wherein the FR-β inducer is a retinoic acid agonist, or is a retinoic acid agonist and a histone deacetylase inhibitor.

5. The method of claim 4 wherein the retinoic acid agonist is all-trans retionic acid (ATRA).

6. The method of claim 4 wherein the histone deacetylase inhibitor is Trichostatin A (TSA).

8. The method of claim 1 wherein the folate-conjugated therapeutic comprises folate associated with one or more therapeutic substances.

9. The method of claim 1 wherein the folate-conjugated therapeutic comprises particles or liposomes.

10. The method of claim 1 wherein the folate-conjugated therapeutic comprises folate-coated liposomes and one or more therapeutic substances.

11. The method of claim 10 wherein one of the therapeutic substances is doxorubicin (DOX).

12. The method of claim 1 wherein the FR-β inducer is administered to the patient concurrently with or before the folate-conjugated therapeutic is administered to the patient.

13. The method of claim 1 further comprising the step of combining said method for treating leukemia with chemotherapy, surgery, radiotherapy, photodynamic therapy, gene therapy, antisense therapy, enzyme prodrug therapy, immunotherapy, fusion toxin therapy, antiangiogenic therapy, or a combination thereof.

14. A pharmaceutical composition comprising a FR-β inducer or a folate-conjugated therapeutic or both a FR-β inducer and a folate-conjugated therapeutic.

15. A kit for use in treating leukemia in a patient, the kit comprising one or more FR-β inducers and one or more folate-conjugated therapeutics.
